(19) United States
(12) Patent Application Publication
(10) Pub. No.: US 2026/0216256 A1
(43) Pub. Date: Jul. 30, 2026

(54) Methods of Treating Skin Inflammation and Related Compositions

(71) Applicant: Pendulum Therapeutics, Inc., San Francisco, CA (US)

(72) Inventors: Marion Leclerc, San Francisco, CA (US); John S. Eid, San Francisco, CA (US)

(21) Appl. No.: 19/158,272

(22) PCT Filed: Feb. 23, 2024
(86) PCT No.: PCT/US2024/017134

§ 371 Date: Aug. 20, 2025

Related U.S. Application Data
(60) Provisional application No. 63/447,871, filed on Feb. 23, 2023.
(60) Provisional application No. 63/451,848, filed on Mar. 13, 2023.
(60) Provisional application No. 63/585,439, filed on Sep. 26, 2023.

(51) Int. Cl. A61K 35/74; A61K 31/191; A61K 31/197; A61K 31/20; A61K 31/366; A61K 31/683; A61P 17/00; A61P 17/06; A61P 29/00

(52) U.S. Cl. CPC A61K 35/74; A61K 31/191; A61K 31/197; A61K 31/20; A61K 31/366; A61K 31/683; A61P 17/00; A61P 17/06; A61P 29/00

(57) ABSTRACT

Provided are methods of treating skin inflammation in a subject in need thereof. The methods comprise administering to the subject a composition comprising Akkermansia muciniphila and/or an Akkermansia muciniphila supernatant or fraction thereof comprising one or more agents that reduce skin inflammation (e.g., gluconic acid, gluconolactone, 5-aminolevulinic acid, azelaic acid, glycero-phosphatidylcholine, glycero-phosphatidylinositol, or any combination thereof), in an amount effective to treat the skin inflammation. According to some embodiments, the composition is administered orally or topically. In certain embodiments, prior to the administering, the subject has been identified as having an inflammatory skin disorder, e.g., actinic keratoses, psoriasis, acne, rosacea, seborrheic dermatitis, eczema, or the like. Also provided are topical formulations comprising an Akkermansia muciniphila supernatant or fraction thereof comprising one or more agents that reduce skin inflammation. According to some embodiments, the topical formulation is a lotion, cream, ointment, gel, paste, aerosol spray, aerosol foam, or transdermal patch.

### CROSS-REFERENCE TO RELATED APPLICATIONS

This application claims the benefit of U.S. Provisional Patent Application No. 63/585,439, filed Sep. 26, 2023, U.S. Provisional Patent Application No. 63/451,848, filed Mar. 13, 2023, and U.S. Provisional Patent Application No. 63/447,871, filed Feb. 23, 2023, which applications are incorporated herein by reference in their entireties.

### SUMMARY

Provided are methods of treating skin inflammation in a subject in need thereof. In certain embodiments, the methods comprise administering to the subject a composition comprising Akkermansia muciniphila, and/or an Akkermansia muciniphila supernatant or fraction thereof comprising one or more agents that reduce skin inflammation (e.g., gluconic acid, gluconolactone, 5-aminolevulinic acid, azelaic acid, glycero-phosphatidylcholine, glycero-phosphatidylinositol, or any combination thereof), in an amount effective to treat the skin inflammation. According to some embodiments, the composition is administered orally or topically. In certain embodiments, prior to the administering, the subject has been identified as having an inflammatory skin disorder, e.g., actinic keratoses, psoriasis, acne, rosacea, seborrheic dermatitis, eczema, or the like. Also provided are topical formulations comprising an Akkermansia muciniphila supernatant or fraction thereof comprising one or more agents that reduce skin inflammation. According to some embodiments, the topical formulation is a lotion, cream, ointment, gel, paste, aerosol spray, aerosol foam, or transdermal patch.

### BRIEF DESCRIPTION OF THE FIGURES

FIG. 1: Before and after photographs of subjects treated with A. muciniphila for inflammatory skin disorders. The upper left and right photographs are before and after photographs (respectively) of a first subject diagnosed with psoriasis. The photographs demonstrate the efficacy of A. muciniphila for treatment of psoriasis. The lower left and right photographs are before and after photographs (respectively) of a second subject diagnosed with eczema. The photographs demonstrate the efficacy of A. muciniphila for treatment of eczema.

FIG. 2: Depiction of an assay employed to assess the effect of treatment of human epidermal keratinocytes with Akkermansia muciniphila supernatant on IL-17A levels.

FIG. 3A-3B: Data demonstrating that A. muciniphila cell free supernatant (CFS) treatment reduces the levels of IL17A and IL23 from THP-1 derived monocytes and HEKa cells under stimulatory condition, respectively. (***~p<0.0001; **~p<0.005; *~p<0.5)

FIG. 4A-4B: Human L-cells were cultured with Akkermansia muciniphila growth medium (4A) or Akkermansia muciniphila CFS (4B). GLP-1 secretion is shown by signal from the anti-GLP-1 polyclonal antibody with Alexa 488 fluorophore.

FIG. 5: Data demonstrating reduction of pro-inflammatory cytokines IL6 and IL1β by Akkermansia supernatant treated THP-1 derived macrophages under LPS induced condition.

FIG. 6: Survey results from 180 subjects treated with Akkermansia muciniphila for +90 days (1 capsule/day-100M active-fluorescent units (AFUs) per capsule). A substantial portion of the subjects reported improvement in their skin.

FIG. 7A-7C: Mass spectrometry data demonstrating the presence of gluconic acid (7A), gluconolactone (7B), and 5-aminolevulinic acid (7C) in Akkermansia muciniphila supernatant.

### DETAILED DESCRIPTION

Before the methods and compositions of the present disclosure are described in greater detail, it is to be understood that the methods and compositions are not limited to particular embodiments described, as such may, of course, vary. It is also to be understood that the terminology used herein is for the purpose of describing particular embodiments only, and is not intended to be limiting, since the scope of the methods and compositions will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the methods and compositions. The upper and lower limits of these smaller ranges may independently be included in the smaller ranges and are also encompassed within the methods and compositions, subject to any specifically excluded limit in the stated range. Where the stated range includes one or both of the limits, ranges excluding either or both of those included limits are also included in the methods and compositions.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the methods and compositions belong. Although any methods and compositions similar or equivalent to those described herein can also be used in the practice or testing of the methods and compositions, representative illustrative methods and compositions are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present methods and compositions are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the methods and compositions, which are, for clarity, described in the context of separate embodiments, may also be provided in combination in a single embodiment. Conversely, various features of the methods and compositions, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present methods and compositions and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order that is logically possible.

### Methods of Treating Skin Inflammation

Aspects of the present disclosure include methods of treating skin inflammation. According to some embodiments, the methods comprise administering to the subject a composition comprising Akkermansia muciniphila and/or an Akkermansia muciniphila supernatant or fraction thereof comprising one or more agents that reduce skin inflammation (e.g., gluconic acid, gluconolactone, 5-aminolevulinic acid, azelaic acid, glycero-phosphatidylcholine, glycero-phosphatidylinositol, or any combination thereof), in an amount effective to treat the skin inflammation. The methods of the present disclosure are based at least in part on the demonstration herein of the presence of skin inflammation-reducing agents in Akkermansia muciniphila supernatant, and that treatment with Akkermansia muciniphila supernatant reduces interleukin 17A (IL-17A) from human keratinocytes and reduces interleukin 23 (IL-23) secretion from human macrophages. IL-17A triggers cellular reactions in the keratinocytes, and also in other cells including neutrophils, endothelial cells, fibroblasts, and osteoclasts. In keratinocytes, the binding of IL-17A to IL-17 receptor (IL-17R) A, IL-17C, or IL-17RD stimulates keratinocyte proliferation. Subsequently, the release of inflammatory mediators and chemokines leads to an inflammatory reaction. IL-23 signaling induces the expression of a unique set of inflammatory genes, engaging type 17 immune responses. This includes the retinoic acid receptor-related orphan receptor-γt (RORγt, encoded by the Rorc gene), a master regulator of type 17 helper T (Th17) cells. The IL-23 signal is critical for maturation and stabilization of the proinflammatory Th17 phenotype. Further details regarding the role of IL-17A and IL-23 in skin inflammation and inflammatory skin diseases can be found in, e.g., Liu et al. (2020) Front Immunol. 11:594735, and elsewhere. Details regarding embodiments of the methods of the present disclosure will now be described.

According to some embodiments, the methods comprise administering to the subject a composition comprising Akkermansia muciniphila. Akkermansia muciniphila is a gram negative, strict anaerobe that can play a role in mucin degradation. Akkermansia muciniphila can serve as a primary fermenter, and in some cases, be combined with any one or more of the secondary fermenters. In certain embodiments, if the composition comprises one or more bacterial strains in addition to the Akkermansia muciniphila, then the Akkermansia muciniphila constitutes at least 50%, at least 60%, at least 70%, at least 80%, at least 90%, or at least 95% of the bacteria in the composition. According to some embodiments, the composition comprises microbes consisting essentially of, or consisting of, the Akkermansia muciniphila.

Individual doses of the composition may include, as to any one of the one or more microbial populations (e.g., Akkermansia muciniphila) included in a given dose, between about 1×10⁷ and 1×10¹⁵ CFUs per dose. In some cases, the administration will be at least 1×10⁷ CFUs of the microbes per dose, at least 1×10⁸ CFUs per dose, at least 1×10⁹ CFUs per dose, at least 1×10¹⁰ CFUs per dose, at least 1×10¹¹ CFUs per dose, at least 1×10¹² CFUs per dose, at least 1×10¹³ CFUs per dose, at least 1×10¹⁴ CFUs per dose, or mores. In some cases, the administration will be at least 1×10⁷ active-fluorescent units (AFUs) per dose, e.g., from about 1×10⁷ to about 1×10⁹ AFUs per dose, e.g., about 1×10⁸ AFUs per dose.

When the methods comprise administering to the subject a composition comprising Akkermansia muciniphila, the composition may be administered via any suitable route of administration. Non-limiting examples of suitable routes of administration include oral and topical administration. For example, when the methods comprise administering to the subject a composition comprising Akkermansia muciniphila, in some instances, the administering is by oral administration. In certain embodiments, when administered orally, the composition is in pill form. Non-limiting examples of such formulations include tablets, capsules, or the like. According to some embodiments, when administered orally, the composition is a food, drink, dietary supplement, food supplement, or food additive.

In certain embodiments, the composition comprises an Akkermansia muciniphila supernatant or fraction thereof comprising one or more agents that reduce skin inflammation. By Akkermansia muciniphila “supernatant” is meant a medium in which Akkermansia muciniphila has been cultured. As demonstrated herein, during culture, Akkermansia muciniphila secretes agents known to reduce skin inflammation (e.g., gluconic acid, gluconolactone, 5-aminolevulinic acid, etc.) into the culture medium. In certain embodiments, a fraction of an Akkermansia muciniphila supernatant comprises, consists essentially of, or consists of, one or more of the one or more agents that reduce skin inflammation. Approaches for obtaining a supernatant fraction of interest are known and include centrifugation, elutriation, chromatography, and the like. Moreover, approaches for determining whether a fraction comprises one or more agents of interest that reduce skin inflammation are available and include, e.g., mass spectrometry, an assay for IL-17A secretion for keratinocytes, an assay for IL-23 secretion from macrophages, and any combination thereof. Non-limiting examples of approaches for performing mass spectrometry and assays for IL-17A and IL-23 secretion are provided in the Experimental section herein.

When the methods comprise administering to the subject a composition comprising an Akkermansia muciniphila supernatant or fraction thereof comprising one or more agents that reduce skin inflammation, the composition may be administered via any suitable route of administration. Non-limiting examples of suitable routes of administration include oral and topical administration. For example, when the methods comprise administering to the subject a composition comprising an Akkermansia muciniphila supernatant or fraction thereof comprising one or more agents that reduce skin inflammation, in some instances, the administering is by topical administration. In certain embodiments, when the composition is administered topically, the composition comprises a lotion, cream, ointment, gel, paste, aerosol spray, aerosol foam, or transdermal patch. Details regarding such compositions formulated for topical administration may be found, e.g., in Mayba et al. (2017) J Cutan Med Surg. 22 (2): 207-212, and elsewhere.

In some instances, the one or more agents that reduce skin inflammation is one or any combination of gluconic acid, gluconolactone, 5-aminolevulinic acid, azelaic acid, glycero-phosphatidylcholine, and glycero-phosphatidylinositol. Gluconic acid (formula C₆H₁₂O₇) and gluconolactone (formula C₆H₁₀O₆) share the same pathway and are part of the same family of molecules, alpha hydroxy acids, which are strong anti-oxidants and known for their anti-aging and soothing properties. 5-aminolevulinic acid (5-ALA) is found in products such as Levulan®, Ameluz® and Gleolan@ and approved for the treatment of actinic keratoses from sun or UV exposure, 10% of which become cancerous. It is used for the treatment of non-melanoma skin cancer and off-label with excellent results for acne. 5-ALA reduces LPS inflammation in macrophages. Azelaic acid is a naturally occurring dicarboxylic acid found in cereal grains like wheat, rye and barley and animal products. It is produced by Malassezia furfur, a yeast that lives on normal skin. Azelaic acid is used to treat acne and rosacea as a 10-20% gel or cream, and also dermatitis. Phosphatidylcholine (PC) is a phospholipid that incorporates choline as a headgroup. It is the major component of biological membranes. Phosphatidylinositol (PI) is a phospholipid with a hydrophobic domain consisting of two fatty acid molecules attached by ester bonds to a three-carbon glycerol moiety in the plane of the plasma membrane. Both PC and PI exhibit reduced levels and have been implicated in inflammatory skin conditions such as psoriasis. See, e.g., Zeng et al. (2017) Gigascience 6 (10): 1-11.

Any of the compositions administered according to the methods of the present disclosure may include additional components, including but not limited to, at least one preservative. In particular embodiments, the composition may contain an effective amount of a preservative. An “effective” amount of a preservative is any amount that preserves or increases the shelf life of the composition beyond what would be obtained if the preservative were not present in the formulation. Examples of such preservatives include, but are not limited to, Vitamin E, Vitamin C, butylatedhydroxyanisole (BHA). butylatedhydroxytoluene (BHT), disodium ethylenediaminetetraacetic acid (EDTA), polyphosphates, citric acid, benzoates, sodium benzoate, sorbates, propionates, and nitrites.

A variety of subjects are treatable according to the methods of the present disclosure. The term “subject” as used herein refers to any living organism, including, but not limited to, humans, nonhuman primates such as chimpanzees and other apes and monkey species; farm animals such as cattle, sheep, pigs, goats and horses; domestic mammals such as dogs and cats; laboratory animals including rodents such as mice, rats, rabbits and guinea pigs, and the like. The term does not denote a particular age or sex. In certain embodiments, the subject is human.

According to some embodiments, the subject (e.g., a human subject) has an inflammatory skin disorder. In some instances, prior to the administering, the subject has been identified as having an inflammatory skin disorder. In certain embodiments, prior to the administering, the subject has been identified as having an inflammatory skin disorder, and the method is for treating the inflammatory skin disorder. According to some embodiments, the inflammatory skin disorder is actinic keratoses, psoriasis, acne, rosacea, seborrheic dermatitis, or allergic contact dermatitis. According to some embodiments, the inflammatory skin disorder is psoriasis. In some instances, the inflammatory skin disorder is eczema. When the inflammatory skin disorder is eczema, in certain embodiments, the eczema is not atopic dermatitis. In other embodiments, when the inflammatory skin disorder is eczema, the eczema is atopic dermatitis.

In certain embodiments, the inflammatory skin disorder is an autoimmune inflammatory skin disorder. Autoimmune inflammatory skin disorders of interest include, but are not limited to, lupus erythematosus, vitiligo, neutrophilic dermatoses, cryopyrin-associated periodic syndrome (CAPS), Familial Mediterranean fever (FMF), or pyrin-associated autoinflammation with neutrophilic dermatosis (PAAND).

According to the methods of the present disclosure, the composition is administered in an amount effective to treat the skin inflammation. By “effective amount” is meant a dosage sufficient to produce a desired result, e.g., an amount sufficient to effect beneficial or desired therapeutic (including prophylactic) results, such as the prevention or a reduction in a symptom of skin inflammation (e.g., a symptom of an inflammatory skin disorder), as compared to a control. In some embodiments, the effective amount is sufficient to slow the progression of, or reduce, one or more symptoms of skin inflammation (e.g., a symptom of an inflammatory skin disorder) selected from erythema, itching, dryness, heat, blistering, and/or the like. According to some embodiments, the effective amount slows the progression of, or reduces, one or more of such symptoms by 10% or more, 20% or more, 30% or more, 40% or more, 50% or more, 60% or more, 70% or more, 80% or more, 90% or more, or 100% or more, as compared to the one or more symptoms in the absence of the administration of the composition.

An effective amount can be administered in one or more administrations. In some cases, the methods comprise administration of multiple doses over a period of time. In certain embodiments, administration may comprise administration of 1, 2, 3, 4, 5, 6 or more doses over the period of a day. In some cases, such daily administration may occur 1 day, 2 days, 3 days, 4 days, 5 days, 6 days or 7 days during a week. In some cases, such weekly administration may occur over the course of 1 week, 2 weeks, 3 weeks, 4 weeks, 6 weeks, 10 weeks, 12 weeks or longer. In some cases, such longer term administration may occur over the course of 1 month, 2 months, 3 months, 4 months, 5, months, 6 months, 7 months, 8 months, 9 months, 10 months, 11 months, 12 months or longer. In some cases administration may be ongoing in order to maintain effects of such treatment, e.g., with the above described administration occurring over the course of 1 year, 2 years, 3 years or longer.

Aspects of the present disclosure further include methods of increasing the level of one or any combination of gluconic acid, gluconolactone, 5-aminolevulinic acid, azelaic acid, glycero-phosphatidylcholine, and glycero-phosphatidylinositol in a subject in need thereof. Such methods comprise administering to the subject a composition comprising Akkermansia muciniphila and/or an Akkermansia muciniphila supernatant or fraction thereof, in an amount effective to increase the level of one or any combination of gluconic acid, gluconolactone, 5-aminolevulinic acid, azelaic acid, glycero-phosphatidylcholine, and glycero-phosphatidylinositol in the subject. In some instances, the composition, dosage, route of administration (e.g., topical or oral) and administration regimen are as described elsewhere herein in the context of treating skin inflammation. In certain embodiments, prior to the administering, the subject has been identified as having an inflammatory skin disorder, e.g., one or any combination of the inflammatory skin disorders described elsewhere herein.

Aspects of the present disclosure further include methods of reducing interleukin 17A (IL-17A) production from keratinocytes in a subject in need thereof, and/or methods of reducing interleukin 23 (IL-23) secretion from macrophages in a subject in need thereof. Such methods comprise administering to the subject a composition comprising Akkermansia muciniphila and/or an Akkermansia muciniphila supernatant or fraction thereof, in an amount effective to reduce IL-17A production from keratinocytes in the subject and/or reduce IL-23 secretion from macrophages in the subject. In certain embodiments, the composition, dosage, route of administration (e.g., topical or oral) and administration regimen are as described elsewhere herein in the context of treating skin inflammation. In certain embodiments, prior to the administering, the subject has been identified as having an inflammatory skin disorder, e.g., one or any combination of the inflammatory skin disorders described elsewhere herein.

### Topical Formulations

Aspects of the present disclosure further include topical formulations. The topical formulations find use in a variety of contexts, including but not limited to, practicing the methods of the present disclosure.

In certain embodiments, a topical formulation of the present disclosure comprises an Akkermansia muciniphila supernatant or fraction thereof comprising one or more agents that reduce skin inflammation, e.g., one or any combination of gluconic acid, gluconolactone, 5-aminolevulinic acid, azelaic acid, glycero-phosphatidylcholine, and glycero-phosphatidylinositol.

According to some embodiments, the topical formulation comprises a lotion, cream, ointment, gel, paste, aerosol spray, aerosol foam, or transdermal patch. Any of the topical formulations of the present disclosure may include additional components, including but not limited to, at least one preservative. Non-limiting examples of preservatives that find use in the topical formulations of the present disclosure include, but are not limited to, Vitamin E, Vitamin C, butylatedhydroxyanisole (BHA). butylatedhydroxytoluene (BHT), disodium ethylenediaminetetraacetic acid (EDTA), polyphosphates, citric acid, benzoates, sodium benzoate, sorbates, propionates, and nitrites.

### Methods of Producing Gluconic Acid, Gluconolactone, 5-Aminolevulinic Acid, Azelaic Acid, Glycero-Phosphatidylcholine, and Glycero-Phosphatidylinositol

Aspects of the present disclosure further include methods of producing one or any combination of gluconic acid, gluconolactone, 5-aminolevulinic acid, azelaic acid, glycero-phosphatidylcholine, and glycero-phosphatidylinositol. In certain embodiments, such methods comprise culturing Akkermansia muciniphila, and harvesting the supernatant of the Akkermansia muciniphila culture. According to some embodiments, the methods further comprise fractionating the supernatant to obtain a fraction of the supernatant comprising one or any combination of the gluconic acid, gluconolactone, 5-aminolevulinic acid, azelaic acid, glycero-phosphatidylcholine, and glycero-phosphatidylinositol. Suitable approaches for fractionating supernatants include centrifugation, elutriation, chromatography, and the like.

The methods of producing one or any combination of gluconic acid, gluconolactone, 5-aminolevulinic acid, azelaic acid, glycero-phosphatidylcholine, and glycero-phosphatidylinositol may further comprise purifying one or any combination of the gluconic acid, gluconolactone, 5-aminolevulinic acid, azelaic acid, glycero-phosphatidylcholine, and glycero-phosphatidylinositol from the supernatant or fraction thereof.

For purposes of completeness, the present disclosure is further defined in the following numbered clauses.

1. A method of treating skin inflammation in a subject in need thereof, the method comprising administering to the subject a composition comprising Akkermansia muciniphila and/or an Akkermansia muciniphila supernatant or fraction thereof comprising one or more agents that reduce skin inflammation, in an amount effective to treat the skin inflammation.

2. The method according to clause 1, wherein the composition comprises Akkermansia muciniphila.

3. The method according to clause 2, wherein if the composition comprises one or more bacterial strains in addition to the Akkermansia muciniphila, then the Akkermansia muciniphila constitutes at least 50%, at least 60%, at least 70%, at least 80%, at least 90%, or at least 95% of the bacteria in the composition.

4. The method according to clause 2, wherein the composition comprises microbes consisting essentially of, or consisting of, the Akkermansia muciniphila.

5. The method according to any one of clauses 2 to 4, wherein the administering is by oral administration.

6. The method according to clause 5, wherein the composition is in pill form.

7. The method according to clause 6, wherein the pill is a tablet or capsule.

8. The method according to clause 5, wherein the composition is a food, drink, dietary supplement, food supplement, or food additive.

9. The method according to clause 1, wherein the composition comprises an Akkermansia muciniphila supernatant or fraction thereof comprising one or more agents that reduce skin inflammation.

10. The method according to clause 9, wherein the administering is by topical administration.

11. The method according to clause 10, wherein the composition comprises a lotion, cream, ointment, gel, paste, aerosol spray, aerosol foam, or transdermal patch.

12. The method according to any one of clauses 9 to 11, wherein the agent that reduces skin inflammation is gluconic acid, gluconolactone, 5-aminolevulinic acid, azelaic acid, glycero-phosphatidylcholine, glycero-phosphatidylinositol, or any combination thereof.

13. The method according to any one of clauses 1 to 12, wherein the composition comprises a preservative.

14. The method according to any one of clauses 1 to 13, wherein prior to the administering, the subject has been identified as having an inflammatory skin disorder.

15. The method according to clause 14, wherein the inflammatory skin disorder is actinic keratoses, psoriasis, acne, rosacea, seborrheic dermatitis, or allergic contact dermatitis.

16. The method according to clause 14, wherein the inflammatory skin disorder is eczema.

17. The method according to clause 16, wherein the eczema is not atopic dermatitis.

18. The method according to clause 16, wherein the eczema is atopic dermatitis.

19. The method according to clause 14, wherein the inflammatory skin disorder is an autoimmune inflammatory skin disorder.

20. The method according to clause 19, wherein the autoimmune inflammatory skin disorder is lupus erythematosus, vitiligo, neutrophilic dermatoses, cryopyrin-associated periodic syndrome (CAPS), Familial Mediterranean fever (FMF), or pyrin-associated autoinflammation with neutrophilic dermatosis (PAAND).

21. A topical formulation comprising an Akkermansia muciniphila supernatant or fraction thereof comprising one or more agents that reduce skin inflammation.

22. The topical formulation of clause 21, wherein the composition comprises a lotion, cream, ointment, gel, paste, aerosol spray, aerosol foam, or transdermal patch.

23. The topical formulation of clause 21 or clause 22, wherein the agent that reduces skin inflammation is gluconic acid, gluconolactone, 5-aminolevulinic acid, azelaic acid, or any combination thereof.

24. The method according to any one of clauses 21 to 23, wherein the topical formulation comprises a preservative.

25. A method of producing one or any combination of gluconic acid, gluconolactone, 5-aminolevulinic acid, azelaic acid, glycero-phosphatidylcholine, and glycero-phosphatidylinositol, the method comprising:
culturing Akkermansia muciniphila; and
harvesting the supernatant of the Akkermansia muciniphila culture.

26. The method according to clause 25, further comprising fractionating the supernatant to obtain a fraction of the supernatant comprising one or any combination of the gluconic acid, gluconolactone, 5-aminolevulinic acid, azelaic acid, glycero-phosphatidylcholine, and glycero-phosphatidylinositol.

27. The method according to clause 25 or clause 26, further comprising purifying one or any combination of the gluconic acid, gluconolactone, 5-aminolevulinic acid, azelaic acid, glycero-phosphatidylcholine, and glycero-phosphatidylinositol, from the supernatant or fraction thereof.

28. A composition comprising the one or any combination of the gluconic acid, gluconolactone, 5-aminolevulinic acid, azelaic acid, glycero-phosphatidylcholine, and glycero-phosphatidylinositol, produced according to the method of any one of clauses 25 to 27.

29. The composition of clause 28, wherein the composition is formulated for topical administration to a subject in need thereof.

30. The composition of clause 29, wherein the composition comprises a lotion, cream, ointment, gel, paste, aerosol spray, aerosol foam, or transdermal patch.

The following examples are offered by way of illustration and not by way of limitation.

### EXPERIMENTAL

### Example 1—Beneficial Effects of A. Muciniphila on Skin Appearance and Related Symptoms

In this example, a subject suffering from skin inflammation is administered an oral composition comprising Akkermansia muciniphila. In some instances, prior to the administering, the subject has been identified as having an inflammatory skin disorder, and the method is for treating the inflammatory skin disorder.

The oral composition comprises between 1×10⁷ and 1×10¹⁵ CFUs of the Akkermansia muciniphila, and one or more doses of the oral composition are administered per day. The delivery form of the oral composition is an enteric-coated (e.g., pH sensitive polymer) capsule or tablet that can protect against stomach acidity and deliver to the ileum/upper colon region of the subject. The enteric coating can be designed to dissolve at a pH greater than about 6.5-7. In some embodiments, the oral composition can be administered as a capsule comprising a powdered Akkermansia muciniphila composition.

Administration of the oral composition comprising Akkermansia muciniphila continues at least until one or more symptoms of the skin inflammation (e.g., one or more symptoms of an inflammatory skin disorder) improves. Symptoms may include erythema, itching, dryness, heat, blistering, and/or the like. The administration may slow the progression of, or reduce, one or more of such symptoms by 10% or more, 20% or more, 30% or more, 40% or more, 50% or more, 60% or more, 70% or more, 80% or more, 90% or more, or 100% or more, as compared to the one or more symptoms in the absence of the administration of the composition. Before and after photos may be taken to demonstrate the improvement in the one or more symptoms.

### Example 2—Efficacy of A. Muciniphila for Treatment of Inflammatory Skin Disorders

Demonstrated in this example is the efficacy of administering A. muciniphila for treatment of inflammatory skin disorders.

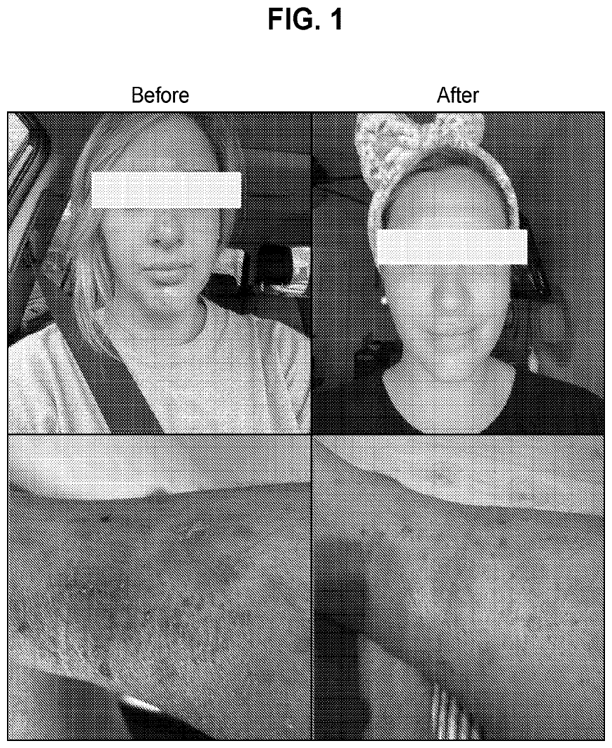

A first subject diagnosed with psoriasis was administered A. muciniphila orally (1 capsule/day—100M active-fluorescent units (AFUs) per capsule) for 2 months. Photographs of the subject were taken before and after treatment. The before and after photographs of this subject are the upper left and right photographs (respectively) of FIG. 1, demonstrating the efficacy of A. muciniphila for treatment of psoriasis.

A second subject diagnosed with eczema was administered A. muciniphila orally (1 capsule/day—100M AFUs per capsule) for 7 weeks. Photographs of the second subject were taken before and after treatment. The before and after photographs of this subject are the lower left and right photographs (respectively) of FIG. 1, demonstrating the efficacy of A. muciniphila for treatment of eczema.

A third subject diagnosed with acne was administered A. muciniphila orally (1 capsule/day—100M AFUs per capsule). A dramatic improvement in skin appearance was observed after four months—including a dramatic reduction of acne, establishing the efficacy of A. muciniphila for treatment of acne.

### Example 3—A. Muciniphila Supernatant Reduces IL-17A Secretion from Human Keratinocytes and IL-23 Secretion from Human Macrophages

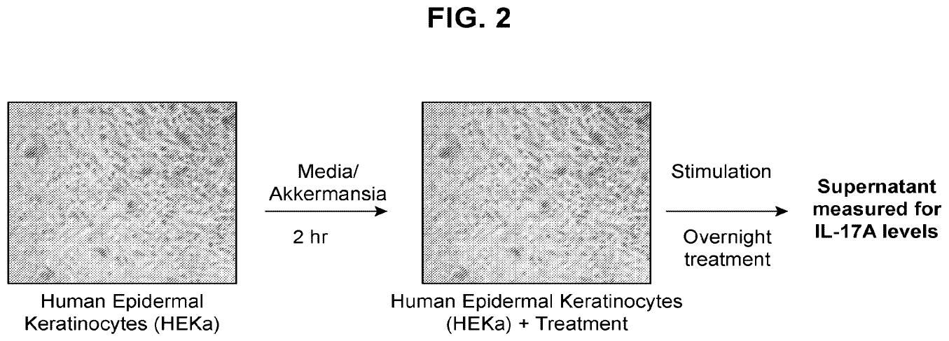

Demonstrated first in this example is that A. muciniphila supernatant reduces IL-17A levels from human keratinocytes. Shown in FIG. 2 is the assay employed to assess the effect of treatment of human epidermal keratinocytes with Akkermansia muciniphila supernatant on IL-17A levels. Briefly, Normal Human Primary Epidermal Keratinocytes (HEKa) were treated with either Akkermansia muciniphila cell free supernatant (CFS) or control culture medium, followed by assessment of IL-17A secretion from the HEKa cells in each condition.

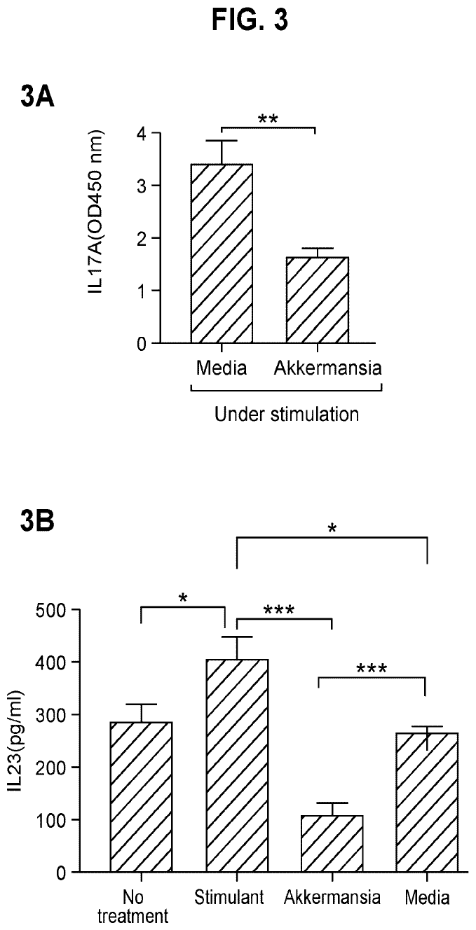

As shown in FIG. 3A, HEKa cells treated with Akkermansia muciniphila CFS secreted significantly less IL-17A than HEKa cells treated with control medium. A similar study was conducted to assess IL-23 secretion from human macrophages. In particular, THP-1 derived human macrophages were treated with either Akkermansia muciniphila CFS or control culture medium, followed by assessment of IL-23 secretion from the human macrophages in each condition. As shown in FIG. 3B, the human macrophages treated with Akkermansia muciniphila supernatant secreted significantly less IL-23 than the human macrophages treated with control medium.

### Methods for Example 3

### Strain, Growth Medium and Conditions

A. muciniphila was grown in a medium having the ingredients and concentrations listed in the following table.

AMUC_OG media recipe

| Component | Amount per liter | |
|---|---|---|
| Sodium Chloride | 4.9 | g |
| Sodium Bicarbonate | 0.4 | g |
| Monopotassium phosphate | 0.04 | g |
| Magnesium Sulfate Heptahydrate | 0.02 | g |
| Potassium Phosphate, Dibasic | 2.04 | g |
| Calcium Chloride | 0.02 | g |
| Dextrose | 2 | g |
| Sodium Phosphate Dibasic | 2.5 | g |
| Cysteine•HCl | 0.5 | g |
| N-Acetylglucosamine | 2 | g |
| HiVeg Special Infusion | 7.5 | g |
| HiVeg Extract #2 | 10 | g |
| Hiveg Peptone #3 | 10 | g |
| AntiFoam | 0.05 | ml |
| D-Biotin | 0.0002 | g |
| Calcium Pantothenate | 0.0026 | g |
| Myoinositol | 0.02 | g |
| p-Aminobenzoic Acid | 0.0005 | g |
| Pyridoxine Hydrochloride | 0.005 | g |
| Riboflavin | 0.0005 | g |
| Thiamine Hydrochloride | 0.01 | g |
| Vitamin B12 (4C) | 0.0002 | g |
| Nicotinic acid | 0.005 | g |

### Supernatant Obtention and Lyophilization

A. muciniphila supernatant was collected approximately 16 days post growth under manufacturing conditions in Fed Batch fermentation settings. The OD₆₀₀ nm at the time of sample collection was ~8 (late exponential to early stationary phase). The culture samples were collected and dispensed into sterile 50 mL Eppendorf conical tubes and stored at −80° C. Prior to mass spectrometry analysis, the Akkermansia muciniphila cultures were thawed and filtered-sterilized by a 0.2 μM filter.

The lyophilization process includes a first cycle at −40° C. (5 min at 500 mTorr), followed by a primary dry step at 20° C. (10 min at 500 mTorr), followed by a second cycle at −40° C. (999 min at 3000 mTorr).

### In Vitro Assays

Normal Human Primary Epidermal Keratinocytes (HEKa) were obtained from ATCC (PCS-200-011). HEKa cells were cultured in Dermal Cell Basal Medium medium supplemented with Keratinocyte Growth Kit, both obtained from ATCC and maintained in an incubator at 37° C. in the presence of 5% CO₂. HEKa cells were treated with 2.5 ng/ml M5 (IL-1α, II-17A, II-22, oncostatin M and TNF-α; PeproTech) for 16 hours at 37° C. to mimic psoriatic condition in vitro.

The human THP-1 monocytic cell line was obtained from ATCC (TIB-202). THP-1 cells were cultured in RPMI-1640 supplemented with 10% fetal bovine serum and 0.05 mM 2-mercaptoethanol and maintained in an incubator at 37° C. in the presence of 5% CO₂. To differentiate into macrophages, THP-1 cells were treated with 500 nM of PMA for 4 hours and allowed to differentiate in fresh culture media for 4 days. THP-1 derived macrophages were treated with LPS (1000 ng/ml) and IFNγ (50 ng/ml) for 16 hours at 37° C. to induce secretion of IL-23.

HEKα cells and THP-1 derived macrophages were treated with either Akkermansia muciniphila supernatant or control media.

### Cytokine Measurement

HEKα and macrophage cell culture supernatants were harvested and the levels of IL-17A (ab119535) and IL-23 (R&D systems D2300B) were measured using ELISA according to the manufacturer's instructions.

### Example 4—A. Muciniphila Supernatant Enhances the Secretion of GLP-1 and Reduces Secretion of the Pro-Inflammatory Cytokines IL6 and IL13

A. muciniphila was grown under anaerobic conditions. In vitro assays were performed on cell free supernatant (CFS) obtained by filter-sterilization. Human L-cells were cultured with Akkermansia CFS and Akkermansia growth medium. Cells were fixed and labeled with anti-GLP1 polyclonal antibody with Alexa488 fluorophore. Human THP-1 monocytic cells were treated with PMA to derive macrophages which were then treated with either Akkermansia CFS or growth media in the presence of a stimulator (LPS) for secretion of IL6 and IL1β. IL6 and IL1β in the cell culture supernatant were quantified by ELISA.

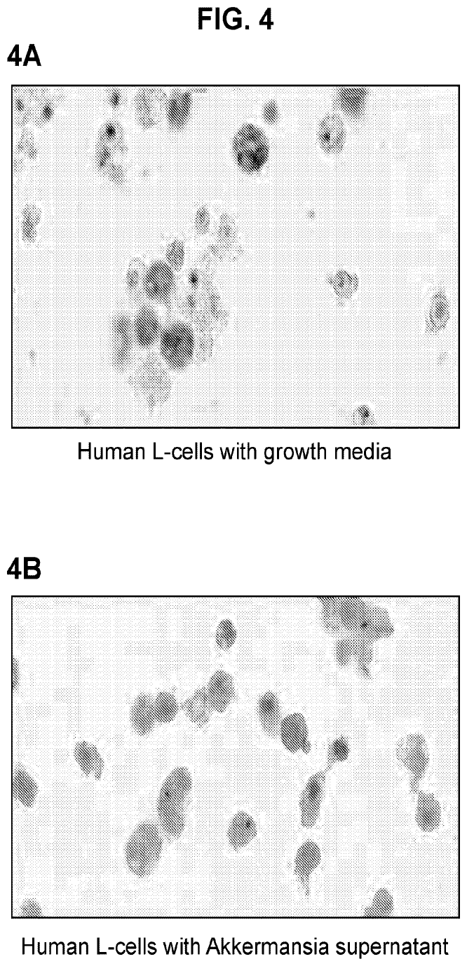

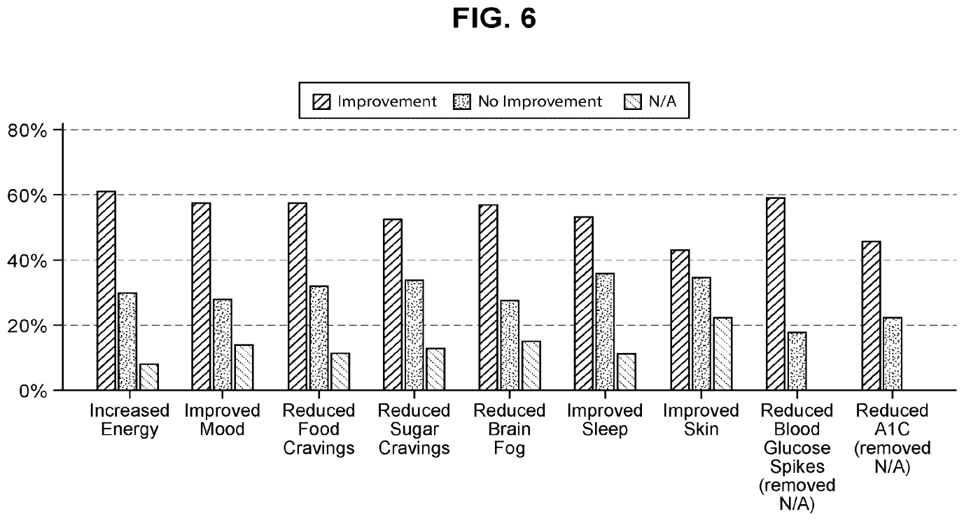

Akkermansia supernatant treated L-cells secreted higher amounts of GLP-1 compared to the growth medium (FIG. 4A-4B). These results are consistent with survey data from 180 subjects taking Akkermansia where 60% and nearly 50% of them experienced reduced blood glucose spikes and reduced A1C, respectively (FIG. 6). Notably, a substantial portion of the subjects reported improvement in their skin.

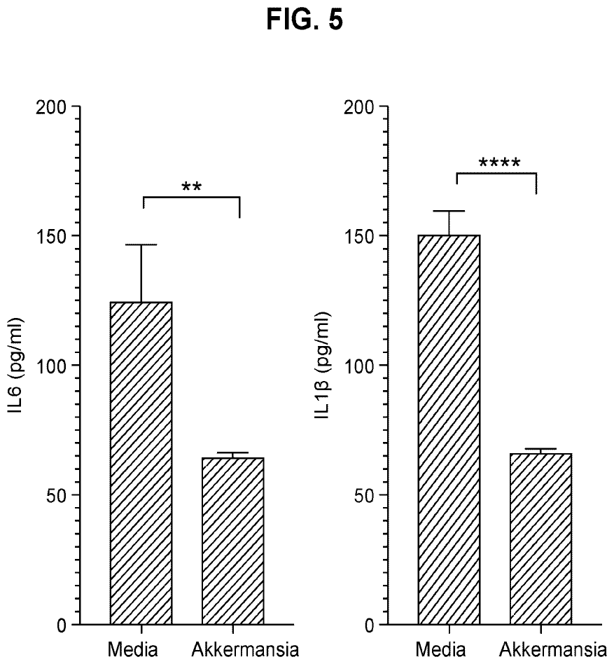

Similarly, the levels of pro-inflammatory cytokines IL6 and IL1β were significantly reduced when LPS stimulated THP-1 derived macrophages were treated with Akkermansia CFS (FIG. 5).

### Example 5—Detection of Skincare Active Agents in A. Muciniphila Supernatant by Mass Spectrometry

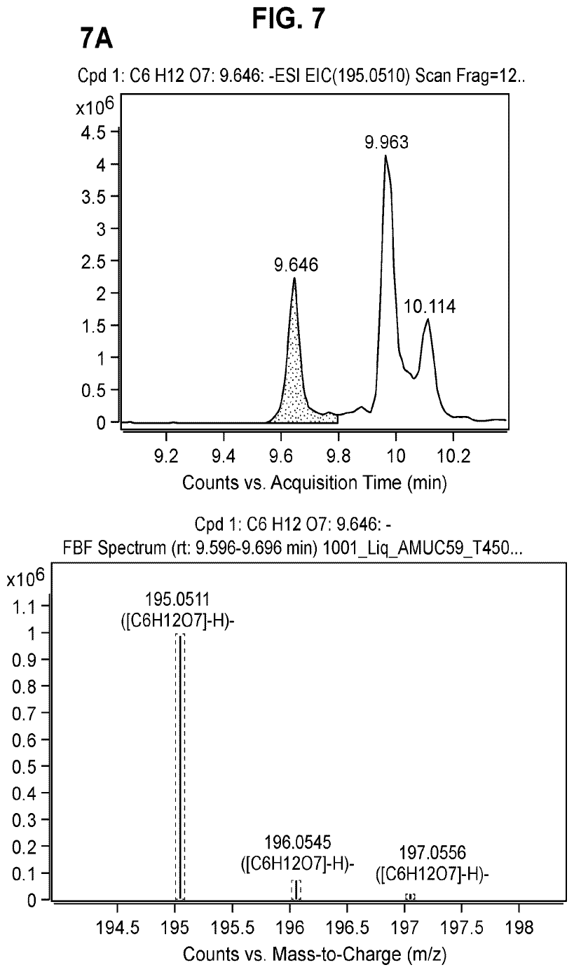

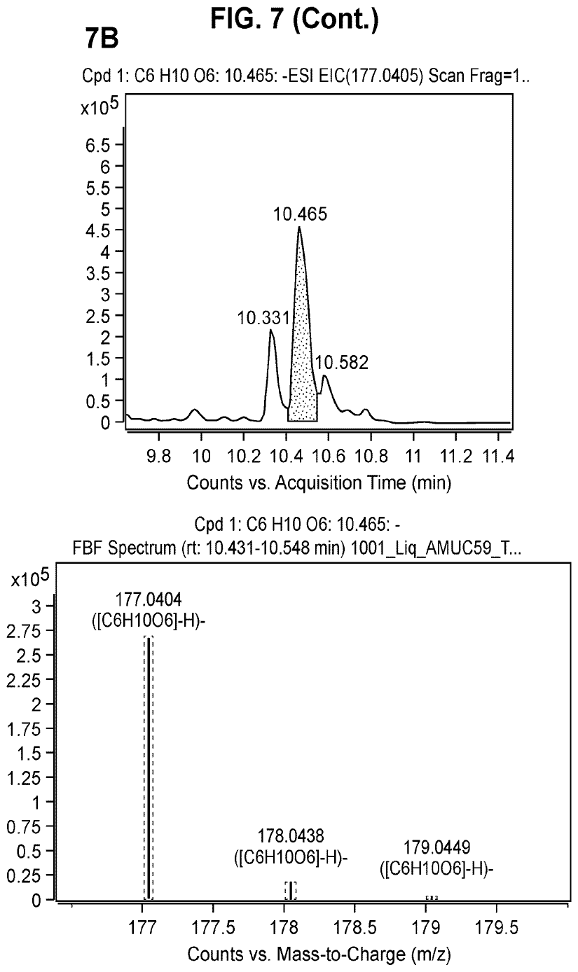

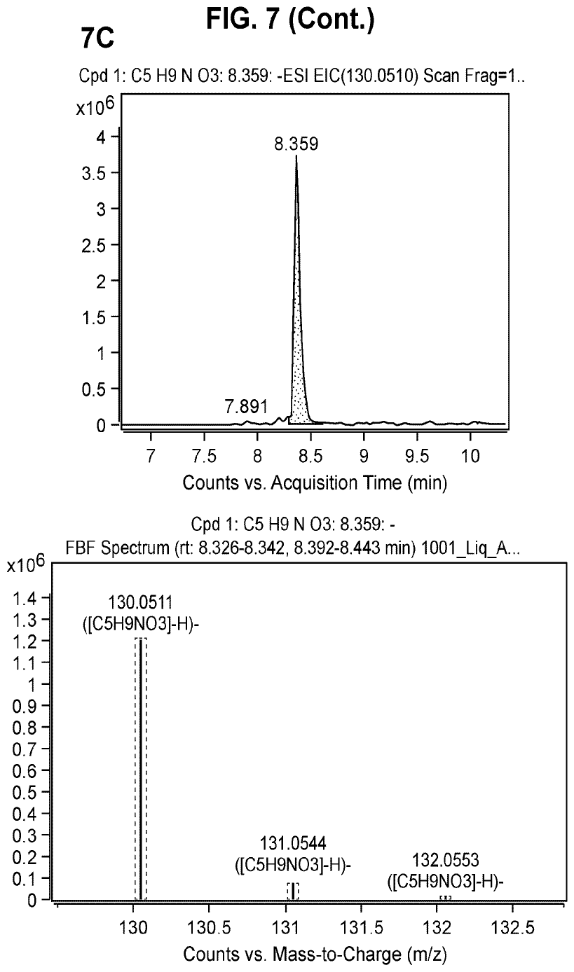

In this example, Akkermansia muciniphila supernatant was analyzed by mass spectrometry. As shown in FIG. 7A-7C, it was determined that the supernatant comprises the skincare active agents gluconic acid, gluconolactone, and 5-aminolevulinic acid, respectively.

### Methods for Example 5

### Methods and Database

An Agilent 6546QTOF was used for untargeted and targeted analysis of metabolites present in Akkermansia muciniphila supernatant. LC methods were used: Reversed phase (RP) was used for polar metabolites; a Hydrophilic Interaction Liquid Chromatography (HILIC) method was used for polar metabolites.
i) Mass profiler MPP and MH Profinder 10.0 were first used to find gluconic acid, gluconolactone and 5-aminolevulinc acid in a non-targeted manner. The ID browser was used for automated ID identification of the metabolites.
ii) the Metlin curated database (>240,000 compounds) was used to build a target metabolite custom database. The targeted compound extraction algorithm used i) theoretical m/z values from formulas as a list of target ions, ii) uses theoretical isotope peak heights/ratios iii) makes use of Retention Time if available. The threshold for mass accuracy detection was 5 ppm.

Compound ID validation: To enable Library Search with METLIN, samples were acquired in MS2 mode and precursor ions were set to fragment at 10, 20, and 40 eV.

### Statistical Analysis

Akkermansia muciniphila growth medium vs 450 h. Triplicate injections and normalization were performed.

### Metabolites Mass, and MS/MS Fractionation Peak

Gluconic acid (formula C₆H₁₂O₇) was detected at a 9.464 acquisition time (see Counts vs Acquisition time in FIG. 7A) and fractionations at m/z 195.0511; 196.0545; 197.0556 (see Counts vs Mass-to-Charge in FIG. 7A).

Gluconolactone (formula C₆H₁₀O₆) was detected at a 10.465 acquisition time (see Counts vs Acquisition time in FIG. 7B) and fractionations at m/z 177.0404; 178.0438; 179.0449 (see Counts vs Mass-to-Charge in FIG. 7B).

5-aminolevulinic acid was detected at a 8.359 acquisition time (see Counts vs Acquisition time in FIG. 7C) and fractionations at m/z 130.0511; 131.0544, 132.0553 (see Counts vs Mass-to-Charge in FIG. 7C).

### Example 6—Efficacy of A. Muciniphila for Treatment of Lesions

A subject diagnosed with Langerhans cell histiocytosis (LCH) was administered A. muciniphila orally (1 capsule/day—100M AFUs per capsule) for 5 months. The subject suffered from lesions associated with the LCH. By the end of 5 months of the oral administration of A. muciniphila, the lesions were completely resolved, establishing the efficacy of A. muciniphila for treatment of lesions associated with Langerhans cell histiocytosis.

### Example 7—A. Muciniphila Produces Glycero-Phosphatidylcholine and Glycero-Phosphatidylinositol

In this example, metabolomics analysis of Akkermansia muciniphila was carried out using the Metabolon platform. A. muciniphila was grown in a medium having the ingredients and concentrations listed in the methods for Example 3, and was collected post-growth under manufacturing conditions. The OD600 nm at the time of sample collection was 1.67 which was at the end of log (early stationary) phase. The culture samples were collected, filtered, and spun down for 6 minutes to collect the pellet. This process was repeated twice. The pellet was then washed in PBS twice and stored. Approximately 20-50 mg of pellet was used to perform the Metabolon studies on the pellet obtained.

Metabolomics analysis of the A. muciniphila pellet determined that the pellet comprised glycero-phosphatidylcholine and glycero-phosphatidylinositol, thus determining that these two phospholipids are produced by A. muciniphila. Assessment of the supernatant for the presence or absence of glycero-phosphatidylcholine determined that this phospholipid was indeed present in the supernatant. The presence or absence of glycero-phosphatidylinositol in the supernatant was not assessed in this study.

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

1. A method of treating skin inflammation in a subject in need thereof, the method comprising administering to the subject a composition comprising Akkermansia muciniphila and/or an Akkermansia muciniphila supernatant or fraction thereof comprising one or more agents that reduce skin inflammation, in an amount effective to treat the skin inflammation.

2. The method according to claim 1, wherein the composition comprises Akkermansia muciniphila.

3. The method according to claim 2, wherein the composition comprises one or more bacterial strains in addition to the Akkermansia muciniphila, then and the Akkermansia muciniphila constitutes at least 50%, at least 60%, at least 70%, at least 80%, at least 90%, or at least 95% of the bacteria in the composition.

4. The method according to claim 2, wherein the composition comprises microbes consisting essentially of, or consisting of, the Akkermansia muciniphila.

5. The method according to claim 1, wherein the administering is by oral administration.

6.-9. (canceled)

10. The method according to claim 1, wherein the administering is by topical administration.

11. The method according to claim 10, wherein the composition comprises a lotion, cream, ointment, gel, paste, aerosol spray, aerosol foam, or transdermal patch.

12. The method according to claim 1, wherein the agent that reduces skin inflammation is gluconic acid, gluconolactone, 5-aminolevulinic acid, azelaic acid, glycero-phosphatidylcholine, glycero-phosphatidylinositol, or any combination thereof.

13. The method according to claim 1, wherein the composition comprises a preservative.

14. The method according to claim 1, wherein prior to the administering, the subject has been identified as having an inflammatory skin disorder.

15. The method according to claim 14, wherein the inflammatory skin disorder is actinic keratoses, psoriasis, acne, rosacea, seborrheic dermatitis, or allergic contact dermatitis.

16. The method according to claim 14, wherein the inflammatory skin disorder is eczema.

17. The method according to claim 16, wherein the eczema is not atopic dermatitis.

18. The method according to claim 16, wherein the eczema is atopic dermatitis.

19. The method according to claim 14, wherein the inflammatory skin disorder is an autoimmune inflammatory skin disorder.

20. The method according to claim 19, wherein the autoimmune inflammatory skin disorder is lupus erythematosus, vitiligo, neutrophilic dermatoses, cryopyrin-associated periodic syndrome (CAPS), Familial Mediterranean fever (FMF), or pyrin-associated autoinflammation with neutrophilic dermatosis (PAAND).

21. A topical formulation comprising an Akkermansia muciniphila supernatant or fraction thereof comprising one or more agents that reduce skin inflammation.

22. The topical formulation of claim 21, wherein the composition comprises a lotion, cream, ointment, gel, paste, aerosol spray, aerosol foam, or transdermal patch.

23. The topical formulation of claim 21, wherein the agent that reduces skin inflammation is gluconic acid, gluconolactone, 5-aminolevulinic acid, azelaic acid, or any combination thereof.

24. (canceled)

25. A method of producing one or any combination of gluconic acid, gluconolactone, 5-aminolevulinic acid, azelaic acid, glycero-phosphatidylcholine, and glycero-phosphatidylinositol, the method comprising:
culturing Akkermansia muciniphila;
harvesting the supernatant of the Akkermansia muciniphila culture; and
purifying one or any combination of the gluconic acid, gluconolactone, 5-aminolevulinic acid, azelaic acid, glycero-phosphatidylcholine, and glycero-phosphatidylinositol, from the supernatant or fraction thereof from the harvested supernatant.

26.-30. (canceled)
